(19) United States
(12) Patent Application Publication
(10) Pub. No.: US 2005/0153367 A1
(43) Pub. Date: Jul. 14, 2005

(54) Method of detecting immune response

(76) Inventors: Samuel Chun-Lap Lo, Kowloon (CN); Man-Sau Wong, Kowloon (CN); Adam Yuen-Yuen Wang, Kowloon (CN)

Correspondence Address: LAW OFFICES OF;JACOBSON HOLMAN, PROFESSIONAL LIMITED LIABILITY COMPANY, 400 SEVENTH STREET, N.W., WASHINGTON, DC, 20004, US

(21) Appl. No.: 10/756,768

(22) Filed: Jan. 14, 2004

(51) Int. Cl. G01N 33/53; G01N 33/567; C12N 5/06

(52) U.S. Cl. 435/7.2; 435/353

(57) ABSTRACT

Transformed immortalized immune cell lines have traditionally been used as effectors for drug screening. Such programs may overlook an important principle: the initiation of a wide spectrum of immunomodulatory responses requires the collateral interactions of macrophage, B cells and T cells. This invention provides methods of detecting immune response of a substance using viable splenocytes

### FIELD OF THE INVENTION

This invention relates to methods of detecting immune response of a compound, particularly in screening compounds for treating diseases.

### BACKGROUND OF THE INVENTION

Transformed immortalized immune cell lines have traditionally been used as effectors for drug screening. For example, the macrophage cell line (RAW 264.7) is one of the most commonly used cell lines in the field of immunomodulatory and anticancer drug screening. Nevertheless, such programs may overlook an important principle: the initiation of a wide spectrum of immunomodulatory responses requires the collateral interactions of macrophages, B cells and T cells. As a result, chemicals and/or drugs that have the potential to act as immunomodulatory medicine may easily fail to induce apoptosis in cancer cell line(s) and/or secretion of anti-cancer cytokines (e.g. TNF-α) in these “classical” screening systems as some essential components of their activation pathways are lacking. Hence, they can not reveal their true potential as immunomodulatory drug(s).

### OBJECTS OF THE INVENTION

Therefore, it is an object of this invention to resolve at least one or more of the problems as set forth in the prior art. As a minimum, it is an object of this invention to provide the public with a useful choice.

### SUMMARY OF THE INVENTION

Accordingly, this invention provides a method of detecting immune response of a substance including the step of incubating the compound with viable splenocytes.

Preferably, viable splenocytes are extracted from rat.

Preferably the substance is incubated with the splenocytes in a buffer at about 20-40° C., more preferably at about 37° C.

Additionally, the substance incubated with viable splenocytes is analyzed by 2-dimensional polyacrylamide gel electrophoresis (2DE), preferably t detect production of at least one of the proteins TNF-α, IFN-γ and iNOS, hoemotic protein LH-2, cytochrome C oxidase polypeptide IV precursor, DNA polymerase beta, Guanine nucleotide-binding protein G, T-cell surface glycoprotein CD5 precursor and alpha-mannosidase II.

Preferably the substance is incubated with the splenocytes in a buffer at pH of about five to nine, more preferably at pH of about seven.

### BRIEF DESCRIPTION OF THE DRAWINGS

Preferred embodiments of the present invention will now be explained by way of example and with reference to the accompany drawings in which:

FIG. 1 shows isolated splenocytes using Wright-Giemsa staining (X 1000);

FIG. 2 shows effect of Rg1 on TNF-α, IFN-γ secretion and iNOS synthesis, with FIGS. 2A, 2B and 2C showing the immunoblot for TNF-α secretion, IFN-γ secretion and iNOS synthesis after Rg1 treatment respectively;

FIG. 3 shows the time course of TNF-α and IFN-γ secretion from splenocytes with and without Rg1 (5 μg/ml) treatment;

FIG. 4 shows the differential protein expression of splenocytes in the presence or absence of Rg1 compared by “Two-in-One gel”. Rg1 treated sample is shown on the left and control sample is shown on the right. 4A shows the sample in the pH range 4-5.5 and 4B shows the sample in the pH range 5.5-7;

FIG. 5 shows the effect of Rg1 on protein expression in splenocytes by summarizing the effect of Rg1 on splenocytes protein expression and possible regulatory mechanism involved in immunomodulatory activities of Rg1 on splenocytes;

### DETAILED DESCRIPTION OF THE PREFERRED EMBODIMENT

This invention is now described by way of example with reference to the figures in the following paragraphs.

Objects, features, and aspects of the present invention are disclosed in or are obvious from the following description. It is to be understood by one of ordinary skill in the art that the present discussion is a description of exemplary embodiments only, and is not intended as limiting the broader aspects of the present invention, which broader aspects are embodied in the exemplary constructions.

In this invention, in contract to the traditional approach of using cell lines, splenocytes primary cultures are used for drug screening. Such splenocytes cultures contain all three immune cells (about 30% B lymphocyte, 60% CD3⁺T lymphocyte and 5% macrophage) necessary to initiate a wide spectrum of immune responses. Such splenocytes may be extracted from any mammal suitable for drug screening, for example rat, dog, cat, mice, guinea pig, cattle, dear, monkeys, and so on.

In order to perform the drug screening function, the splenocytes have to be viable. The term “viable” here means the splenocytes are still living for which current technologies may be capable of doing so. Such viable cells will not be stained by trypan blue in a one to one mixture of cells and 0.4% w/v trypan blue solution. The viable splenocytes may be removed from the animal with known procedures, and a suitable example will be described in the following section. Non-viable cells cannot be used as effectors in the screening platform as they will not be able to be simulated by the drug candidates and/or interacted with each other effectively. Viable splenocytes usually can be cultivated satisfactorily in most culture media commonly used for animal cell culture work. However, splenocytes cannot stand extreme temperature ranges (i.e. lower than 20° C. and higher than 40° C.). Optimal temperature for viable splenocytes is 37° C. Further, viability of splenocytes will also be decreased if the cultivating medium is in extreme pH (i.e. higher than pH 9 and lower than pH 5).

The compound to be detected, usually in the form of a solution, may then be applied to the viable splenocytes through various methods. For instance, the compound (free from endotoxin contamination) may be added to the viable splenocytes directly. This will of course vary with different compounds to be tested but the addition of compounds to splenocytes is not the theme of this invention.

The use of viable splen cytes in detecting the immune response of Ginseng Rg1 will be detailed in the following paragraphs.

### EXAMPLE

Highly purified Rg1, one of the main saponins in ginseng has been shown to inhibit the growth of cancer cells. It stimulates biochemical synthesis of DNA, protein and lipid in animal tissue. Rg1 was also shown to act as an immunomodulating agent which leads to an increase in nitric oxide production in endothelial cells after Rg1 induction, an increase in T-helper activities and production of cytokine IL-1 and natural killer cell. Other properties include selectively enhanced proliferation of lymphocytes and production of IL-2. It was hypothesized that the ginsenosides' anticarcinogenic effect may correlate to the apoptotic effects initiated either by caspase 3 and or reactive oxygen species. Despite the long list of research that study the effects of ginseng, its molecular mechanisms of actions remain largely unknown.

Materials and Methods

Animals

Young male Sprague Dawley rats between 8 and 10 wk of age weighing 200 gm were provided by the animal holding center of the Department of Applied Biology and Chemical Technology, The Hong Kong Polytechnic University. All studies were conducted according to the principles and procedures detailed in the most recent publication of the NIH Guide for the Care and Use of Laboratory Animals. Animal ethnics approval has been obtained from the Animal Ethnics Sub-committee of the Hong Kong Polytechnic University.

Preparation of Rat Splenocytes

Rats were placed under deep ether anesthesia and exsanguinated via the abdominal aorta. Spleen was removed by aseptic techniques before washed twice in 10 ml of RPMI-1640 (Gibco, USA) medium containing 1000 U/ml penicillin-streptomycin (PS) (Gibco, USA). Spleens were then cut into small pieces before being sucked up and down with a plunger in a plastic syringe. Single cell suspension was prepared and placed into the same medium containing 1000 U/ml PS, and 5% acid citric dextrose. Splenocytes were then isolated by gradient centrifugation by layering the single cell suspension onto Ficoll-Paque Plus gradient (Amersham Bioscience, H.K. Ltd.) and spun at 400 g for 35 min at mom temperature. The splenocytes-containing fraction was collected. Erythrocytes were lyzed by adding four volumes of ice-cold ammonium chloride solution (pH 7.4) at room temperature for 5 min and splenocytes were washed twice in plain medium. Lymphocytes were stained with Wright-Giema stain before examination under light microscope. Cell viability and number were examined after trypan blue staining under the light microscope. Splenocytes (1×10⁶) were cultured in complete RPMI-1640 medium containing 1000 U/ml PS, supplemented with 2 g/L sodium carbonate. Cells were re-suspended in the same medium with 10% FCS (Gibc , USA) and subjected to various treatments after 24 h incubation.

Stimulation of Splenocytes with Rg1

Rg1 (a commonly used dosage of 5 μg/ml) was added to splenocytes in PBS buffer in 6-well flat-bottomed plates (Nunc Maxisorp, USA) in a 5% CO₂ incubator at 37° C. Samples of conditioned media from the incubation mixture were harvested at varying time points (from 12-72 hr). These samples were kept at −80° C. until use. No sample was kept for more than 2 months. Further, global protein expression in samples of Rg1 treated splenocytes (at 12 h and 24 hr intervals) were also performed using 2-DE analysis. Cells incubated with PBS for 24 hr were used as control.

Western Blotting and Immuno-Detection

Supernatants of splenocytes cell culture with and without Rg1 incubation were harvested at different time points and used for the detection of TNF-α and IFN-γ. Similarly, splenocyte lysate was used for the detection of expression of iNOS. The protein content of the supernatant/lysate was determined by the Bradford method (Bio-Rad Laboratory, USA). For each analysis, 350 μg of protein was analyzed by a 12.5% reducing SDS-PAGE gel ran at a constant voltage (150 V) for 1 hr. Proteins were transferred onto a nitrocellulose membranes (Millipore, USA). After washing and blocking, these membranes were probed with either rabbit anti-rat iNOS (transduction Laboratories, USA), or rabbit anti-rat TNF-α (PropTech, USA) or rabbit anti-rat IFNγ (PropTech, USA) at a dilution of 1:500. After incubating at mom temperature for 2 hours, these membranes were washed before incubating with 1:20,000 diluted goat anti-rabbit horseradish peroxidase (HRP) conjugated IgG (Sigma, USA). Positive binding was detected using the SuperSignal West Pico Chemiluminescent Kit (Pierce, USA) and the exposed X-ray films (Kodak) were scanned with a Molecular Dynamics Densitometer (Rio-Rad Laboratories, USA).

ELISA Assay

IFN-γ as well as TNF-α ELISA assays were performed using the OptEIA™ rat IFN-γ and TNF-α kits (Pharmigen, USA). Splenocytes with or without Rg1 stimulation were tested. In these kits, monoclonal anti-rat IFN-γ or TNF-α antibody was used as th primary antibody and biotinlated monoclonal anti-rat INF-γ or TNF-α antibody was used as the secondary antibody. After the addition of avidin-HRP conjugate, color was developed by the addition of 2,2-Azin-bis-3-thylbenzthiazoline-6-sulfonic acid and the color change was monitored at an absorbance of 405 nm with a Microplate Reader (Bio-Rad Laboratories, USA). The amount of INF-γ and TNF-α were calculated by comparing with the standard.

2-DE Analysis of Splenocyte Lysates

Sample Preparation

Splenocytes on culture dishes were rinsed once with RPMI-1640 after incubation with or without Rg1. Subsequently, 0.5 g/L trypsin containing 0.2 g/L EDTA was added and allowed to incubate for 3 min. Cells were then collected by centrifugation at 1,000 g for 5 min and washed with PBS (pH 7.2) twice. Splenocytes were then lysed with a minimal amount of lysis buffer (usually 200 μl) containing 4% Triton X-100, 9 M urea and 1 mM PMSF for 10 min on ice. The supernatant was collected by centrifugation at 3,000 g for 10 min and protein contents were measured by a modified Bradford method using a protein assay kit by Bio-Rad Laboratories, USA. Cell lysates from different animals with the same treatment were pooled together at a protein ratio of 1:1 and was used for 2-DE analysis. 120 μg of splenocytes lysate [in a buffer containing 9 M Urea, 2% w/v Triton X-100, 0.5% w/v DDT (Sigma, USA), 0.4% v/v IPG buffer 4-7 (Amersham Pharmacia Biotech, Uppsala, Sweden) and trace amount of bromophenol blue to a final volume of 300 μl] was used for in-gel sample rehydration.

2-DE

Samples were applied onto IPG strips pH 4-7 (3×170 mm, Bio-Rad Laboratories, USA). Rehydration and subsequent isoelectric focusing was performed using either a Protean IEF Cell (Bio-Rad Laboratories, USA) or an IPGphor (Amersham Biosciences, USA). Rehydration was performed at room temperature for 16 hr at 50 V in the IPG strip holder covered with low viscosity paraffin oil. Isoelectric focusing was then performed using 500 V (1 hr), 1000 V (1 hr), 4000 V (2 hr) and 8000 V (2 hr) sequentially. Strips were then equilibrated in 50 mM Tris-HCl equilibration buffer, pH 6.8, containing 1% (w/v) DTT (Sigma, USA), 9 M urea, 30% v/v glycerol, 2% w/v SDS and a trace amount of bromophenol blue for 10 min. After equilibration, IPG strips were transferred onto a 12.5% SDS-PAGE gels (1600×1600×1 mm) for the second dimension separation ran at a constant current of 30 mA per gel in room temperature in a Protean VI electrophoresis tank (Bio-Rad, USA). Subsequently, the gels were silver-stained with a method compatible with MALDI-TOF analysis. Silver-stained gels were then scanned with a molecular Dynamics Densitometer (Bio-Rad Laboratories, USA.). The resulting 2-DE patterns were analyzed using the software Melanie III (Bio-Rad Laboratories, USA). An estimation of the relative quantitative changes was made on the basis of the change in percentage of volume among silver-stained protein spots. Spot changes of interest were tested on multiple gels for reproducibility.

Protein Visualization with Silver Staining

Silver staining was performed using a mass spectrometry compatible protocol as recommended by Bruker Deltonics (USA), manufacturer of our MALDI-TOF mass spectrometer. Briefly, a gel was fixed initially in a fixative solution containing 50% v/v methanol, 12% v/v acetic acid, 0.0375% v/v formaldehyde for 2 hours. The gel was subsequently washed with 50% methanol for another 20 minutes. The washing step was repeated twice. The gel was then oxidized in a solution containing 0.05% di-sodium thiosulphate for 1 min followed by washing with milli-Q water (3 times, each for 20 minutes). The gel was then incubated in 0.2% silver nitrate containing 0.0375% formaldehyde for 20 minutes. The gel was again washed with milli-Q water for 3 times, each for 20 minutes. Subsequently, the gel was developed in 15% sodium carbonate solution containing 0.0375% formaldehyde and 0.004% disodium thiosulphate until the gel reached a desirable amount of staining. The process should not be longer than 10 minutes. Finally, the reaction was stopped with 25% methanol containing 12% acetic acid.

Protein Identification

Differentially expressed proteins from different treatment were located on the 2-DE gels either by image analysis with the software Melanie III (Bio-Rad, USA) or directly compared using the “Two-in-one” gel method as described previously (Wang, A. Y. Y., Cheung, B. Y., Wong, M. S., Lo, S. C. L. “Two-in-one” gel for spot matching after two-dimensional electrophoresis.” Proteomics 2003, 3, 580-583). Several differentially expre ssed protein spots were chosen and identified by peptide mass fingerprinting using a MALDI-TOF-MS (Autoflex, Bruker Daltonics, Germany). Gel plugs containing the differentially expressed protein spots were cut out from the 2-DE gels and trypsin digestion was performed (using a protocol recommended by Bruker Daltonics, Germany which was a modification of a method described previously by Shevchenko et al. (Shevchenko A, Wilm M, Vorm O, Mann M. Mass spectrometric sequencing of proteins silver-stained polyacrylamide gels. Anal. Chem. 68: 850-8, 1996.). Briefly, g 1 plugs were cut into small pieces and washed with milli-Q water twice. They were then washed with 25 mM ammonium bicarbonate in 50% acetonitrile. Cysteine residues in the samples were reduced with 10 mM DTT and derivatized by treatment with 55 mM iodoacetamide. After acetonitrile dehydration and drying, samples were digested overnight with trypsin (5 ng/μl trypsin in 25 mM ammonium bicarbonate) at 37° C. The digested sample was extracted by sonication with 50% acetonitrile/1% trifluroacetic acid. Supernatant containing the digested peptides was collected. Digested peptides (0.5 μl to 1.5 μl) was mixed with 1-2 μl of α-cyano-4-hydroxycinnamic acid (10 mg/ml in 50% acetonitrile, 0.1% trifluroacetic acid) matrix and 100 fM adrenocorticotropic hormone (ATCH) fragment 18-39 as the internal standard. The instrument was operated in the positive ion reflectron mode at 20 kV accelerating voltage. Resulting peptide mass fingerprints were searched against Swiss-Prot using the MASCOT search engine from Matrix Science. All searches were performed using a mass window between 1 and 100 kDa with a mass tolerance of ±100 ppm. One miss cleavage per sample was allowed and cysteines were assumed to be carbamidomethylated and methionine in oxidation form.

Results

Splenocytes Isolation

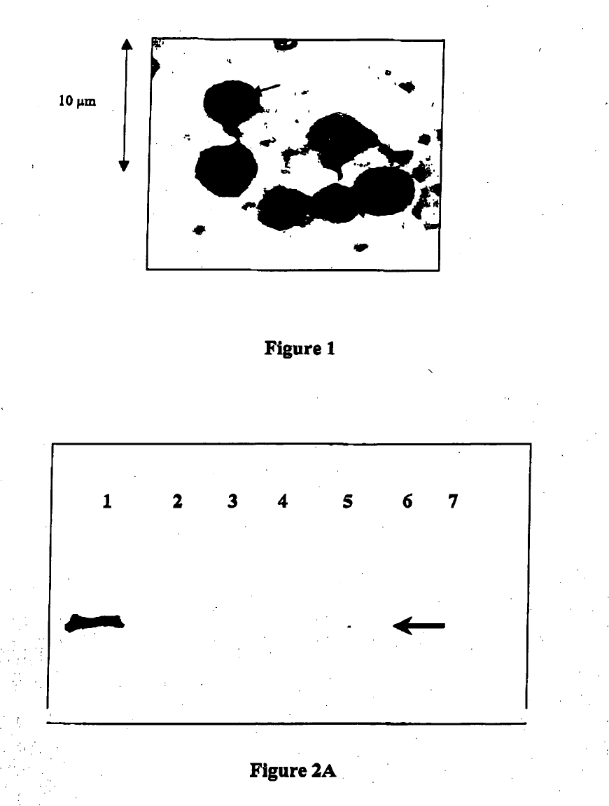

Splenocytes were prepared as described in the Methods section and cells were examined after Wright-Giemsa staining. With a Leica Q500MC Image Processing and Analysis System (Leica, Germany), images of splenocytes (large and small lymphocytes) isolated are shown in FIG. 1. No red cell contamination was seen. Trypan blue staining confirmed that over 95% of cells isolated were viable. As both large and small lymphocytes were present, this preparation can be used as the effectors for studying the immunomodulatory activities of Rg1.

Effect of Rg1 on Splenocytes in Terms of TNF-α, IFN-γ and iNOS Production

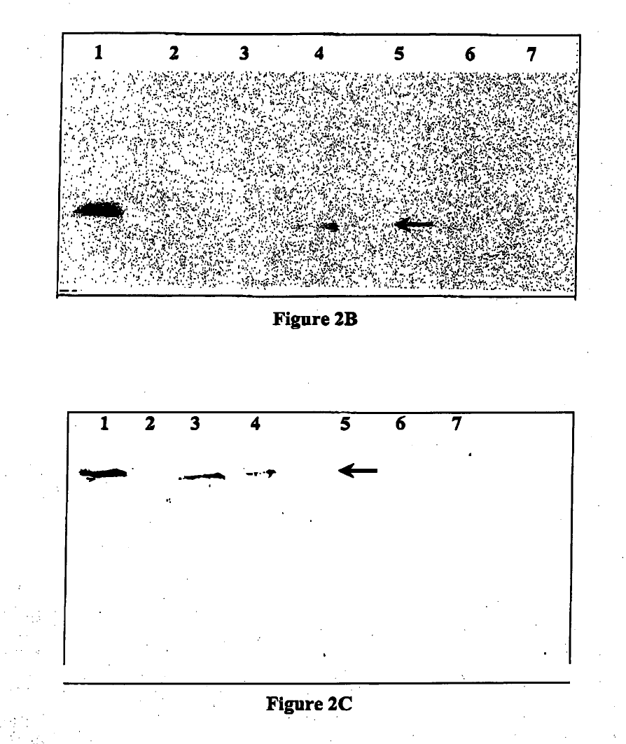

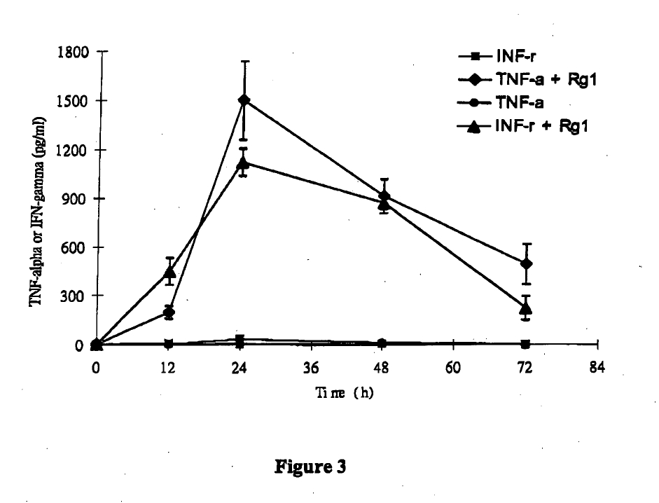

After the successful preparation of viable splenocytes, Rg1 was added as a stimulant to study possible responses of these splenocytes. Western blotting was performed to detect the secretion of TNF-α and IFN-γ in the culture medium. Expression of iNOS was also tested in the splenocyte lysate. Most pharmacological studies of Rg1 used a concentration of 5-20 μg/ml. In our hands, preliminary studies using 5 μg/ml of Rg1 leads to positive outcome in the TNF-α and IFN-γ experiments. Therefore, 5 μg/ml of Rg1 was used in ur experiments. Results of TNF-α, IFN-γ and iNOS Western blotting upon the induction of Rg1 at different time intervals are shown in FIG. 2 (2A is for TNF-α, 2B is for IFNγ and 2C is for iNOS). Splenocytes without Rg1 treatment were used as control for comparison. Results showed that neither TNF-α, nor IFN-γ nor iNOS was detected in either the cell culture medium or the splenocyte lysate during 72 hours of incubation in the control. On the other hand, when stimulated with 5 μg/ml Rg1, the two cytokines and iNOS can be detected. TNF-α was detected both at 24 hr and 48 hr intervals while IFN-γ was detected at 24 hr. Inducible NOS was detected at 12 hr and 24 hr after incubation with Rg1. Subsequently, quantitative documentation of these anticancer cytokines (TNF-α and IFN-γ) was performed using ELISA on serial samples of the culture medium. Each condition was assayed in triplicate and the results are shown in FIG. 3. The secretion patterns of both cytokines are very similar after induction. Quantities of these cytokines increased more than 1000 folds after Rg1 induction and their levels reached a maximum at 24 hr of incubation. After 24 hr., both cytokine secretion levels started to decrease with increase in incubation time. This result is also consistent with the results from Western blot where the highest TNF-α and IFN-γ secretion is found at 24 hours after Rg1 induction.

Effect of Rg1 on Protein Expression

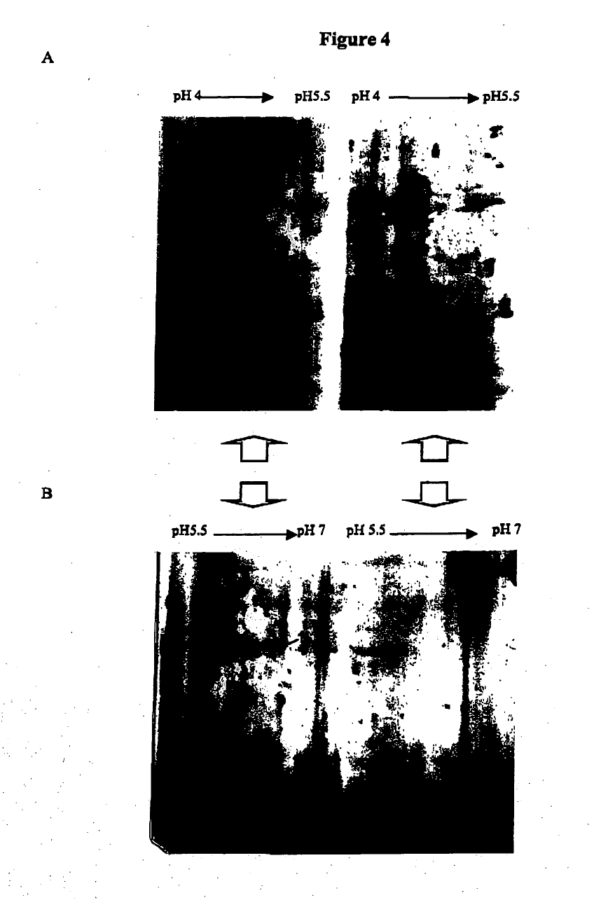

Since Rg1 was found to induce TNF-α and IFN-γ production after 24 hrs of incubation, the effect of Rg1 on the splenocyte proteome after 24 hr of incubation was investigated. Samples with the same treatment were pooled at a protein ratio of 1:1 and used for 2-DE analysis as described in Materials and Methods. All samples were collected after 24 hr of incubation either in PBS or PBS with Rg1. Sample incubated with PBS was used as control for comparison. To facilitate identification of differentially expressed protein, a “Two-in-One” gel method was performed as previously described (Wang et al. 2003). Briefly, the first dimension IEF of samples (with or without Rg1 incubation) was performed separately in different Protean IEF cell but concurrently at the same time. After the IEF was finished, the IEF stripes were cut into equal halves. Halves of the IEF strips that were corresponding to the same pH ranges but ran with different samples (with or without Rg1 incubation) were put side-by-side on top of the second dimension SDS-PAGE prepared in a Protean VI electrophoresis apparatus. The resulting gels of control and Rg1 treated samples were compared after silver staining using Melanin III. FIG. 4 dem nstrated the differences in protein expression between the normal sample and Rg1 treated sample. About 102 protein spots were detected from one half of the gel ran with normal sample and 122 protein spots were detected on the other half of the gel ran with Rg1 treated sample.

Protein Identification

MALDI-TOF-MS was used to identify differentially expressed proteins after in-gel-trypsin digestion. Seven protein spots that were differentially expressed were successfully identified by MASCOT search engine. Six proteins were up-regulated and they included cytochrome C oxidase, homeotic protein LH-2, T-cell surface glycoprotein CD5, DNA polymerase β, α-mannosidase II and guanine nucleotide binding protein G. The other protein was found to be down-regulated and it was identified as hypothetical anti-proliferative factor. Details of the 7 identified proteins are summarized in Table 1.

TABLE I
Identity of differentially expressed protein spots from splenocytes.

| Spot | | Accession | Theoretical | % Sequence | |
|---|---|---|---|---|---|
| No. | Protein name | No. | Mᵣ (kDa)/pI | coverage | Regulation |
| 1 | Homeotic protein | A47179 | 47.38/8.94 | 14% | Up- |
| | LH-2 | | | | regulated |
| 2 | Cytochrome C | NP_058898 | 19.50/9.45 | 26% | Up- |
| | oxidase polypeptide | | | | regulated |
| | IV precursor | | | | |
| 3 | DNA polymerase | AAA41900 | 38.17/8.69 | 20% | Up- |
| | beta | | | | regulated |
| 4 | Guanine nucleotide- | P82471 | 41.45/5.58 | 15% | Up- |
| | binding protein G | | | | regulated |
| 5 | T-cell surface | P51882; | 53.40/8.86 | 14% | Up- |
| | glycoprotein CD5 | Q63098 | | | regulated |
| | precursor | | | | |
| 6 | Alpha-mannosidase | P28494 | 56.70/6.20 | 11% | Up- |
| | II | | | | regulated |
| 7 | Hypothetical anti- | I53276 | 17.74/8.39 | 33% | Down- |
| | proliferative factor | | | | regulated |

Discussion

Rat splenocytes consist of macrophages, T-cells and B-cells. This population of cells was used as effectors to test for the presence of immunomodulatory activities of Rg1. With Western blotting and ELISA, it was found that Rg1 stimulated the production of TNF-α, IFN-γ and iNOS from splenocytes. TNF-α and IFN-γ production reached their peaks at 24 hr after Rg1 induction. Our results are similar to those reported by Gao et al. (Gao H., Wang, F., Lien, E. J., Trousdale M. D. Pharmaceutical Research. 1996, 13, 1196-200), whereby the highest amount of TNF-α and IFN-γ production were detected at 24 hr and 48 hr after induction with a crude preparation of Panax notoginseng on lymphocytes isolated from mouse spleens. In our experiment, a shorter time was needed for the production of TNF-α and IFN-γ to reach their peak values. It might be due to the fact that purified Rg1 instead of a crude preparation was used in our study.

On another front, the mode of action of Rg1 on a molecular level may have been revealed, which may not be discovered in other studies. Differential protein expression in splenocytes as induced by Rg1 was studied. Based on the direct comparison of protein expression from “Two-in-One” gels, significant changes in protein expression between the normal and Rg1 treated sample was found. 87 proteins were found to be up-regulated while another 38 protein spots were found to be down-regulated. Yet another 38 spots did not have any significant apparent changes.

Differentially expressed proteins were selected and identified using MALDI-TOF-MS after trypsin digestion. Seven proteins were successfully identified in which six were up-regulated proteins. They were cytochrome C oxidase, homeotic protein LH-2, T-cell surface glycoprotein CDS, DNA polymerase beta, alpha mannosidase II and guanine nucleotide binding protein G. The remaining protein spot (hypothetical anti-proliferative factor) was found to be down regulated. From the literature, it is known that DNA polymerase-β is related to DNA synthesis. Its up-regulation indicates that Rg1 stimulate DNA synthesis. Whether this stimulation mediates through a steroid hormone-dependent or independent pathway is currently unknown. Rg1 stimulatory effects on DNA synthesis are also observed in rat and mouse bone marrow cells. On the other hand, another 2 up-regulated proteins: homeotic protein LH-2 and the T-cell surface glycoprotein CD5 are known to be involved in B-cell and T-cell proliferation. Homeotic protein LH-2 was reported as an early marker of B-lymphocyte differentiation and also involved in the control of B cell differentiation (Xu, Y., Baldassare, M., Fisher, P., Rathbun, G., Oltz, E. M., Yancop ulos, G. D., Jessell, T. M., Alt, F. W. Proc Natl. cad Sci USA., 1993, 90, 227-31). T-cell surface glycoprotein CD5, a trans-membrane protein, is known to act as a receptor in regulating T-cell proliferation (Vermeer, L. A., de Boer, N. K., Bucci, C., Bos, N. A., Kroese, F. G., Alberti, S. Eur. J. Immunol. 1994, 24, 585-92). Another 2 upregulated proteins that were identified: Cytochrome C oxidase and the alpha-mannosidase II might be involved in cellular metabolism processes. Cytochrome C oxidase is one of the mitochondria membrane enzymes participated in oxidative phosphorylation of the respiratory chain (Kadenbach, B. Biochim. Biophys. Acta. 2003, 1604, 77-94). As ATP is one of the end products of respiratory process, the synthesis of ATP probably increased when cytochrome C is increased. It is known that high ATP content inside the cell contributes to energy supply for cell growth and proliferation. It also participates in Transmembrane signaling pathway for cAMP production (Frizzell, R. A. Am J Respir Crit Care Med. 1995, 151, S54-8). Alpha-mannosidase II is a Golgi membrane protein that controls conversion of mannose to complex N-glycon. It may participate in the lysosomes synthesis as proteins destined to the lysosomes are covalently modified in the Glogi body by the addition of mannose-6-phosphate. However, its exact functions are not clear.

Guanine nucleotide binding protein G is another up-regulated protein which is a membrane-associated protein that couples many types of membrane receptors to various second messenger systems (Gilman, A. G. Ann. Rev. Biochem. 1987, 56, 615-49). The β- and γ-subunits are hydrophobic integral membrane protein that anchor the α subunit, a peripheral protein, to a plasma membrane. It is known that one component of G protein Gsα•GTP component is capable of binding to adenylyl cyclase, which stimulate the production of cAMP (Lania, A., Mantovani, G., Spada, A. Eur. J. Endocrin. 2001, 145, 543-559). It was reported that Rg1 could increase intracellular cAMP and cGMP in aged animals, resulting in the expression of interleukin 2 (IL-2) and splenocytes proliferation (Liu M., Zhang J. T. Yao Xue Xue Bao, 1996, 31, 95-100). Thus, the up-regulation of guanine nucleotide binding protein G may imply a corresponding increase in cAMP level which in turn induced a corresponding increase in IL-2 expression and splenocytes proliferation. Hypothetical anti-proliferative factor, although its real function is not yet established, it is believed to inhibit cellular proliferation. Hence, down-regulation of this protein after Rg1 treatment indicates an increased chance of cellular proliferation.

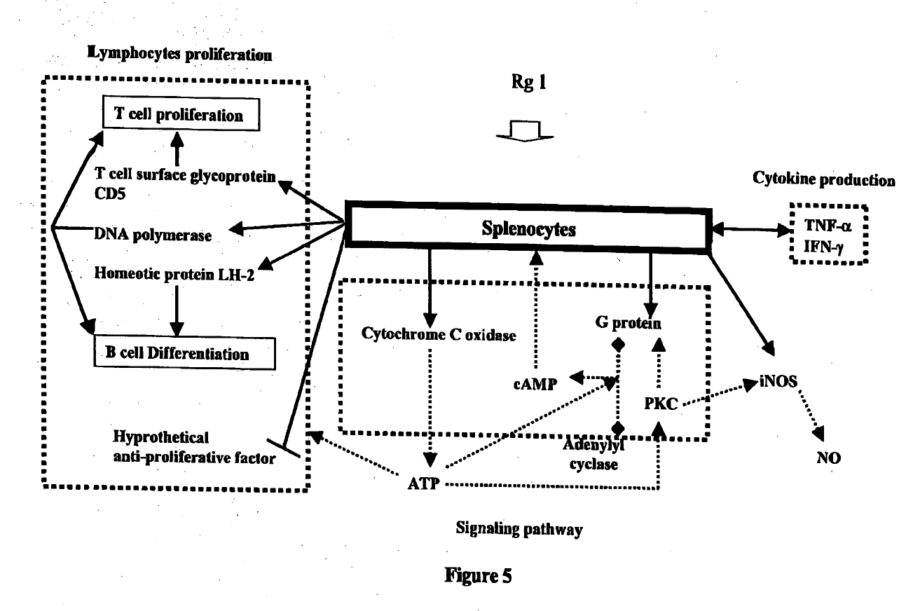

The above results also showed that iNOS was detected at 12 hr and 24 hr after Rg1 treatment. Inducible NOS is known to produce nitric oxide that plays important roles in diverse physiological process such as vasodilatation, inhibition on platelet aggregation and neurotransmission. Nitric oxide is also known to act as a cytotoxic mediator and contributes to the antimicrobial and tumoricidal activity of the macrophages [36]. Thus the up-regulation of iNOS can be used to evaluate antimicrobial activity of cells. Production of this enzyme might be regulated by the cytokines (TNF-α and IFN-γ). Karupiah et al. reported the inhibition of viral replication by IFN-γ induced NOS (Karupiah, G., Xie, Q. W., Buller, R. M., Nathan, C., Duarte, C., MacMicking, J. D. Science 1993, 261, 1445-48). These researchers suggested that the induction of NOS is necessary and sufficient for a substantial antiviral effect of IFN-γ. It was also well documented that IFN-γ could act synergistically with TNF-α to promote gene expression of iNOS. Further, the increase of cytochrome C oxidase and ATP after Rg1 treatment may also increase the production of iNOS. Protein kinase C (PKC) activity is essential for iNOS expression, where PKC requires ATP in the process of binding to membrane receptor. It is also known that PKC-mediated signaling stimulates the activity of G protein. Thus, the increase in iNOS will also leads to increase in the amount of IL-2, TNF-α and IFN-γ. The possible interactions of the 7 identified proteins as well as IL-2, TNF-α, IFN-γ and iNOS in bringing out immunomodulatory effects are summarized in FIG. 5.

To conclude, immunomodulation is a complex activity that involves interaction between various proteins and cellular components. The use of splenocytes to screen for Rg1 immunomodulatory activity has proved to be successful in understanding the mechanisms involved in Rg1 immunomodulation. This invention may provide a framework for large-scale screening of TCM for immunomodulation as well as new drugs in cancer treatment.

While the preferred embodiment of the present invention has been described in detail by the examples, it is apparent that modifications and adaptations of the present invention will occur to those skilled in the art. Furthermore, the embodiments of the present invention shall not be interpreted to be restricted by the examples or figures only. It is to be expressly understood, however, that such modifications and adaptations are within the scope of the present invention, as set forth in the following claims. For instance, features illustrated or described as part of one embodiment can be used on another embodiment to yield a still further embodiment. Thus, it is intended that the present invention cover such modifications and variations as come within the scope of the claims and their equivalents.

1. A method of detecting working mechanism of a substance on an organism including the step of incubating the compound with viable splenocytes.

2. The method of claim 1, wherein the viable splenocytes are extracted from rat.

3. The method of claim 1, wherein the substance is incubated with the splenocytes in a buffer at about 20-40° C.

4. The method of claim 3, wherein the substance is incubated with the splenocytes in a buffer at about 37° C.

5. The method of claim 1 further including the step of analyzing the substance incubated with viable splenocytes by 2-dimensional polyacrylamide gel electrophoresis.

6. The method of claim 5 further including the steps of detecting production of at least one of the proteins and/or its precursors and/or its breakdown products: TNF-α, IFN-γ and iNOS, hoemotic protein LH-2, cytochrome C oxidase polypeptide IV precursor, DNA polymerase beta, Guanine nucleotide-binding protein G. T-cell surface glycoprotein CD5 precursor and alpha-mannosidase II.

7. The method of claim 1, wherein the substance is incubated with the splenocytes in a buffer at a pH of about five to nine.

8. The method of claim 7, wherein the substance is incubated with the splenocytes in a buffer at a pH of about seven.

9. The method of claim 1, wherein the working mechanism is immune response.
